(19) United States
(12) Patent Application Publication
(10) Pub. No.: US 2026/0174511 A1
(43) Pub. Date: Jun. 25, 2026

(54) SURGICAL INSTRUMENT ADAPTER AND SURGICAL ROBOT

(71) Applicant: KAWASAKI JUKOGYO KABUSHIKI KAISHA, Kobe-shi, Hyogo (JP)

(72) Inventors: Fumiya MATSUMOTO, Kobe-shi (JP); Yasuhiro MIYAMOTO, Kobe-shi (JP); Tetsushi ITO, Kobe-shi (JP); Kaoru TAKAHASHI, Kobe-shi (JP)

(73) Assignee: KAWASAKI JUKOGYO KABUSHIKI KAISHA, Kobe-shi, Hyogo (JP)

(21) Appl. No.: 19/128,202

(22) PCT Filed: Oct. 4, 2023
(86) PCT No.: PCT/JP2023/036244

§ 371 Date: May 8, 2025

(30) Foreign Application Priority Data
Nov. 10, 2022 (JP) 2022-180181

(51) Int. Cl. A61B 34/30

(52) U.S. Cl. CPC A61B 34/30; A61B 2034/305

(57) ABSTRACT

In a surgical instrument adapter (300), a first driven portion (320) includes a lever (321) to be rotated by a first drive force, and a roller (322) connected to the lever (321), contacting an operation handle (402), and operable to move while rotating along a surface (402a) of the operation handle (402).

### TECHNICAL FIELD

The present disclosure relates to a surgical instrument adapter and a surgical robot.

### BACKGROUND ART

Conventionally, a surgical instrument adapter for connecting a manual surgical instrument to a robot arm is known. For example, U.S. Patent Application Publication No. 2016/0249993 discloses a surgical instrument adapter for attaching a manual surgical instrument including an operation handle operated by an operator to a robot arm. In U.S. Patent Application Publication No. 2016/0249993, an openable and closable end effector is disposed at a distal end of the manual surgical instrument. The surgical instrument adapter disclosed in U.S. Patent Application Publication No. 2016/0249993 includes a holding member, a mechanical finger member, and a linear actuator. The holding member holds the manual surgical instrument. With the manual surgical instrument held by the holding member, the mechanical finger member contacts the operation handle. The mechanical finger member has a prismatic shape that contacts the operation handle. The linear actuator linearly moves the mechanical finger member such that the prismatic finger member slides on a surface of the operation handle. Thus, the finger member rotates the operation handle. Consequently, the end effector of the manual surgical instrument closes.

### PRIOR ART

### Patent Document

Patent Document 1: U.S. Patent Application Publication No. 2016/0249993

### SUMMARY OF THE INVENTION

However, in U.S. Patent Application Publication No. 2016/0249993, the prismatic finger member rotates the operation handle while sliding on the surface of the operation handle. Therefore, a frictional force acting between the prismatic finger member and the operation handle is relatively large. Consequently, the finger member may not be able to rotate the operation handle smoothly.

The present disclosure is intended to solve the above problem. The present disclosure aims to provide a surgical instrument adapter and a surgical robot each capable of smoothly rotating an operation handle of a manual surgical instrument.

In order to attain the aforementioned object, a surgical instrument adapter according to a first aspect of the present disclosure is a surgical instrument adapter operable to connect a manual surgical instrument to a robot arm, and includes an interface including a first rotor to be rotationally driven by a first drive force transmitted from a first drive in the robot arm, a first driven portion to rotate an operation handle of the manual surgical instrument, and a first drive force transmission mechanism to transmit the first drive force from the first rotor to the first driven portion. The first driven portion includes a lever to be rotated by the first drive force, and a roller connected to the lever, contacting the operation handle, and operable to move while rotating along a surface of the operation handle.

In the surgical instrument adapter according to the first aspect of the present disclosure, as described above, the first driven portion includes the lever to be rotated by the first drive force, and the roller connected to the lever, contacting the operation handle, and operable to move while rotating along the surface of the operation handle. Accordingly, the roller of the first driven portion moves while rotating along the surface of the operation handle, and thus a frictional force acting between the roller and the operation handle is relatively small. Therefore, the first driven portion can smoothly rotate the operation handle of the manual surgical instrument.

A surgical robot according to a second aspect of the present disclosure includes a robot arm including a drive, and a surgical instrument adapter to operably connect a manual surgical instrument to the robot arm. The surgical instrument adapter includes an interface including a rotor to be rotationally driven by a drive force transmitted from the drive in the robot arm, a driven portion to rotate an operation handle of the manual surgical instrument, and a drive force transmission mechanism to transmit the drive force from the rotor to the driven portion. The driven portion includes a lever to be rotated by the drive force, and a roller connected to the lever, contacting the operation handle, and operable to move while rotating along a surface of the operation handle.

In the surgical robot according to the second aspect of the present disclosure, as described above, the driven portion includes the lever to be rotated by the drive force, and the roller connected to the lever, contacting the operation handle, and operable to move while rotating along the surface of the operation handle. Accordingly, the roller of the driven portion moves while rotating along the surface of the operation handle, and thus a frictional force acting between the roller and the operation handle is relatively small. Therefore, it is possible to provide the surgical robot capable of smoothly rotating the operation handle of the manual surgical instrument.

According to the present disclosure, as described above, the operation handle of the manual surgical instrument can be rotated smoothly.

### BRIEF DESCRIPTION OF THE DRAWINGS

FIG. 1 is a diagram showing the configuration of a robotic surgical system according to an embodiment.

FIG. 2 is a diagram showing the configuration of a medical cart according to the embodiment.

FIG. 3 is a diagram showing the configuration of a robot arm according to the embodiment.

FIG. 4 is a diagram showing a pair of forceps.

FIG. 5 is a perspective view showing the configuration of an arm operation unit according to the embodiment.

FIG. 6 is a perspective view showing a state in which an adapter and a medical instrument are removed from a drive of the robot arm according to the embodiment.

FIG. 7 is a perspective view of the adapter and the surgical instrument according to the embodiment, as viewed from the Y2 direction side.

FIG. 8 is a control block diagram of a surgical robot according to the embodiment.

FIG. 9 is a control block diagram of the robot arm according to the embodiment.

FIG. 10 is a control block diagram of the medial cart and a positioner according to the embodiment.

FIG. 11 is a diagram showing a manual surgical instrument.

FIG. 12 is a diagram showing a state in which the manual surgical instrument is attached to a surgical instrument adapter according to the embodiment.

FIG. 13 is a diagram showing a state in which an operation handle disposed in the surgical instrument adapter according to the embodiment is rotated to the A2 side.

FIG. 14 is a perspective view of the surgical instrument adapter according to the embodiment, as viewed from the Y2 direction side.

FIG. 15 is a diagram showing the operation handle and a first driven portion according to the embodiment.

FIG. 16 is a diagram showing the first driven portion according to the embodiment.

FIG. 17 is a perspective view showing a first drive force transmission mechanism according to the embodiment.

FIG. 18 is a perspective view showing a second drive force transmission mechanism according to the embodiment.

FIG. 19 is a diagram showing a state in which the operation handle disposed in the surgical instrument adapter according to the embodiment is rotated to the Al side.

FIG. 20 is a diagram showing a first driven portion according to a modified example.

### MODES FOR CARRYING OUT THE INVENTION

An embodiment embodying the present disclosure is hereinafter described on the basis of the drawings.

### Configuration of Robotic Surgical System

The configuration of a robotic surgical system 100 according to this embodiment is now described. The robotic surgical system 100 includes a surgical robot 1 and a remote control apparatus 2.

In this specification, the longitudinal direction of a surgical instrument 4 is defined as a Z direction, as shown in FIG. 3. The distal end side of the surgical instrument 4 is defined as a Z1 side, and the proximal end side of the surgical instrument 4 is defined as a Z2 side. A direction perpendicular to the Z direction is defined as an X direction. A direction perpendicular to the Z direction and the X direction is defined as a Y direction.

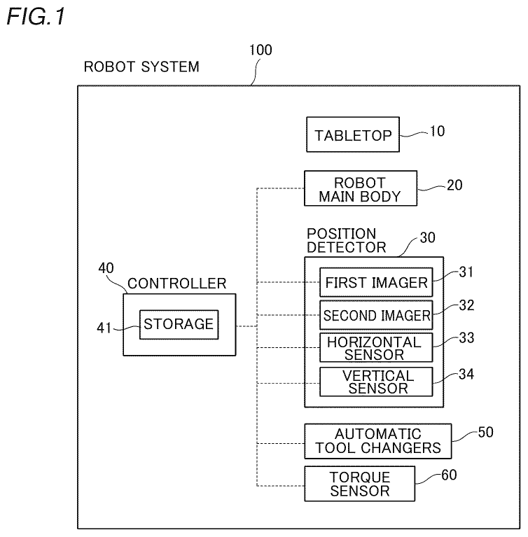

As shown in FIG. 1, the surgical robot 1 is arranged in an operating room. The remote control apparatus 2 is spaced apart from the surgical robot 1. An operator such as a doctor inputs a command to the remote control apparatus 2 to cause the surgical robot 1 to perform a desired operation. The remote control apparatus 2 transmits the input command to the surgical robot 1. The surgical robot 1 operates based on the received command.

The surgical robot 1 is arranged in the operating room that is a sterilized sterile field.

### Configuration of Surgical Robot

As shown in FIG. 1, the surgical robot 1 includes a medical cart 3, a positioner 40, an arm base 50, a plurality of robot arms 60, and arm operation units 80.

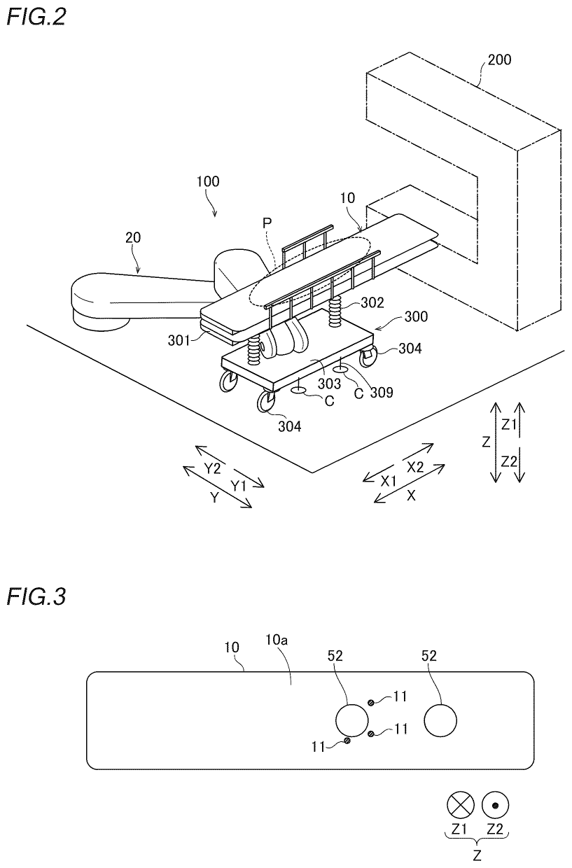

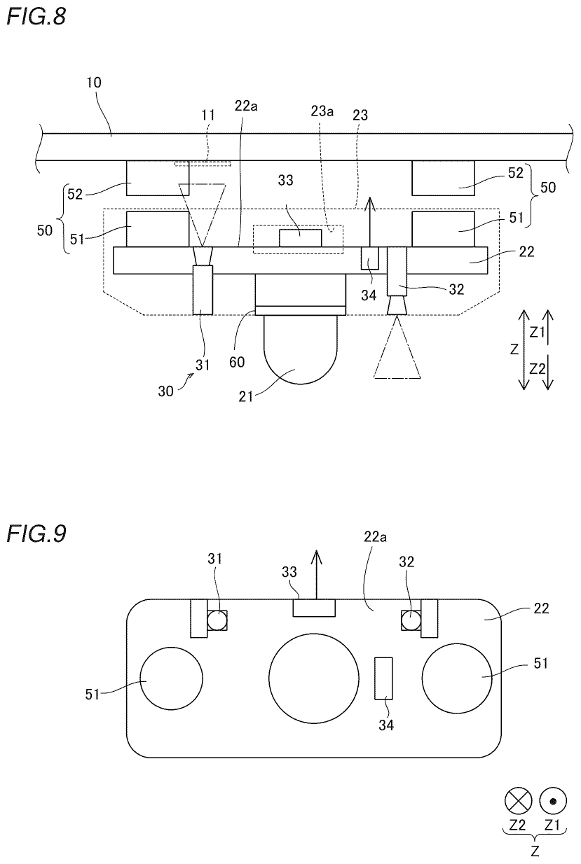

As shown in FIG. 2, the medical cart 3 moves the positioner 40. A cart positioner operation unit 35 includes an input 33. The input 33 receives operations to move the positioner 40, the arm base 50, and the plurality of robot arms 60 or change their postures mainly in order to prepare for surgery before the surgery. The cart positioner operation unit 35 includes an operation handle 34, and a stabilizer 34c and an electric cylinder 34d shown in FIG. 8. The input 33 includes a display 33a. The display 33a is a liquid crystal panel, for example.

As shown in FIG. 2, the cart positioner operation unit 35 is supported by a cart positioner operation support 36 at the rear of the medical cart 3, and the medical cart 3 or the positioner 40 is moved by operating the cart positioner operation unit 35. The cart positioner operation unit 35 includes the input 33 and the operation handle 34. The input 33 includes the display 33a, a joystick 33b, and an enable switch 33c. The joystick 33b is arranged in the vicinity of or adjacent to the input 33 of the cart positioner operation unit 35.

The positioner 40 is moved three-dimensionally by selecting an operation mode displayed on the input 33 and operating the joystick 33b.

The enable switch 33c is arranged in the vicinity of or adjacent to the joystick 33b of the cart positioner operation unit 35. The enable switch 33c enables or disables movement of the positioner 40. When the joystick 33b is operated while the enable switch 33c is pressed to enable movement of the positioner 40, the positioner 40 is moved.

The operation handle 34 is arranged in the vicinity of the display 33a of the cart positioner operation unit 35. The operation handle 34 includes a throttle 34a that is gripped and twisted by an operator such as a nurse or a technician to operate movement of the medical cart 3.

Specifically, the operation handle 34 is arranged below the input 33. As the throttle 34a is twisted from the near side to the far side, the medical cart 3 moves forward. As the throttle 34a is twisted from the far side to the near side, the medical cart 3 moves rearward. The speed of the medical cart 3 is changed according to a twisting amount of the throttle 34a. The operation handle 34 is rotatable to the left and right shown by an R direction, and the medical cart 3 is turned with rotation of the operation handle 34.

An enable switch 34b for enabling or disabling movement of the medical cart 3 is provided on the operation handle 34 of the cart positioner operation unit 35. When the throttle 34a of the operation handle 34 is operated while the enable switch 34b is pressed to enable movement of the medical cart 3, the medical cart 3 is moved.

As shown in FIG. 1, the positioner 40 includes a 7-axis articulated robot, for example. The positioner 40 is arranged on the medical cart 3. The positioner 40 adjusts the position of the arm base 50. The positioner 40 moves the position of the arm base 50 three-dimensionally.

The positioner 40 includes a base 41 and a plurality of links 42 coupled to the base 41. The plurality of links 42 are coupled to each other by joints 43.

The arm base 50 is attached to a distal end of the positioner 40. A proximal end of each of the plurality of robot arms 60 is attached to the arm base 50. Each of the plurality of robot arms 60 is able to take a folded and stored posture. The arm base 50 and the plurality of robot arms 60 are covered with sterile drapes and used. Moreover, each of the robot arms 60 supports the surgical instrument 4.

A status indicator 53 and an arm status indicator 54 that are shown in FIG. 8 are provided on the arm base 50. The status indicator 53 indicates the status of the robotic surgical system 100. The arm status indicator 54 indicates the statuses of the robot arms 60.

The plurality of robot arms 60 are arranged. Specifically, four robot arms 60a, 60b, 60c, and 60d are arranged. The robot arms 60a, 60b, 60c, and 60d have the Same or similar configurations as each other.

As shown in FIG. 3, each robot arm 60 includes an arm portion 61, a first link 72, a second link 73, and a translation mechanism 70. The robot arm 60 have JT1, JT2, JT3, JT4, JT5, JT6, and JT7 axes as rotation axes, and a JT8 axis as a linear motion axis. The JT1 to JT7 axes are rotation axes of joints 64 of the arm portion 61. The JT7 axis is the rotation axis of the first link 72. The JT8 axis is a linear motion axis along which the translation mechanism 70 moves the second link 73 relative to the first link 72 in the Z direction. The arm portion 61 includes a base 62, links 63, and the joints 64.

The arm portion 61 includes a 7-axis articulated robot arm. The first link 72 is arranged at a distal end of the arm portion 61. An arm operation unit 80 described below is attached to the second link 73. The translation mechanism 70 is arranged between the first link 72 and the second link 73. A holder 71 that holds the surgical instrument 4 is arranged on the second link 73.

The surgical instrument 4 is attached to a distal end of each of the plurality of robot arms 60. The surgical instrument 4 includes a replaceable instrument, an endoscope 6 to capture an image of a surgical site, and a pivot position setting instrument to set a pivot position PP, for example. The surgical instrument 4 as an instrument includes a driven unit 4a, a pair of forceps 4b, and a shaft 4c.

As shown in FIG. 1, the endoscope 6 is attached to the distal end of one of the plurality of robot arms 60, such as the robot arm 60c, and the surgical instrument 4 other than the endoscope 6 is attached to the distal end of each of the remaining robot arms 60a, 60b, and 60d, for example. The endoscope 6 is attached to one of two robot arms 60b and 60c arranged in the center among the four robot arms 60 arranged adjacent to each other.

### Configuration Of Instrument

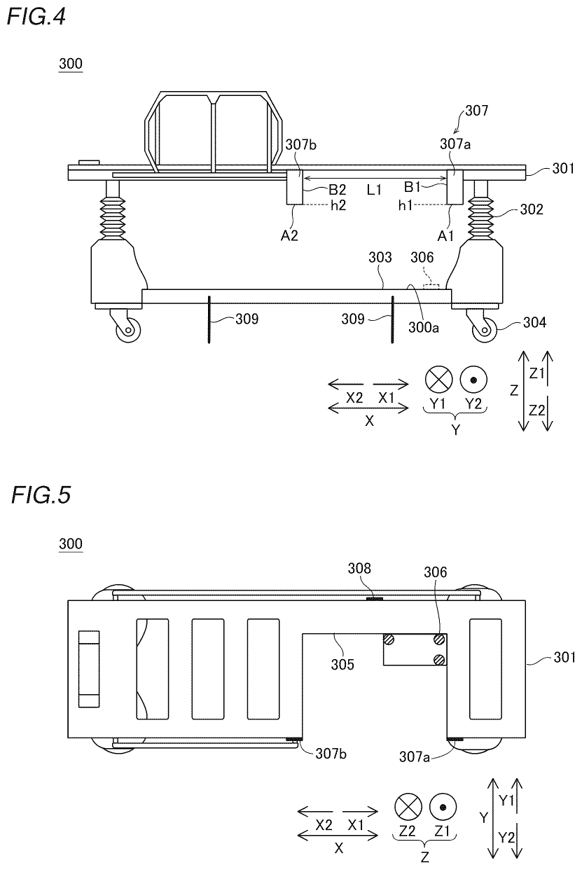

As shown in FIG. 4, the pair of forceps 4b is provided at a distal end of the instrument, for example. As instruments having joints, in addition to the pair of forceps 4b, a pair of scissors, a grasper, a needle holder, a microdissector, a stable applier, a tacker, a suction cleaning tool, a snare wire, a clip applier, etc. are arranged at the distal end of the instrument. At the distal end of the instrument, a cutting blade, a cautery probe, a washer, a catheter, a suction orifice, etc. are arranged as instruments having no joint.

The pair of forceps 4b includes a first support 4e and a second support 4f. The first support 4e supports the proximal end sides of jaw members 104a and 104b such that the jaw members 104a and 104b are rotatable around a JT11 axis. The second support 4f supports the proximal end side of the first support 4e such that the first support 4e is rotatable around a JT10 axis. The shaft 4c rotates around a JT9 axis. The jaw members 104a and 104b pivot around the JT11 axis to open and close.

### Configuration of Arm Operation Unit

As shown in FIG. 5, the arm operation unit 80 is attached to the robot arm 60 to operate the robot arm 60. Specifically, the arm operation unit 80 is attached to the second link 73.

The arm operation unit 80 includes an enable switch 81, a joystick 82, and linear switches 83, a mode switching button 84, a mode indicator 84a, a pivot button 85, and an adjustment button 86.

The enable switch 81 enables or disables movement of the robot arm 60 in response to the joystick 82 and the linear switches 83. When the enable switch 81 is pressed by an operator such as a nurse or an assistant grasping the arm operation unit 80, movement of the surgical instrument 4 by the robot arm 60 is enabled.

The joystick 82 is an operation tool to control movement of the surgical instrument 4 by the robot arm 60. The joystick 82 controls a moving direction and a moving speed of the robot arm 60. The robot arm 60 is moved in accordance with a tilting direction and a tilting angle of the joystick 82.

The linear switches 83 are switches to move the surgical instrument 4 in the Z direction, which is the longitudinal direction of the surgical instrument 4. The linear switches 83 include a linear switch 83a to move the surgical instrument 4 in a direction in which the surgical instrument 4 is inserted into a patient P, and a linear switch 83b to move the surgical instrument 4 in a direction in which the surgical instrument 4 is moved away from the patient P. Both the linear switch 83a and the linear switch 83b are push-button switches.

The mode switching button 84 is a push-button switch to switch between a mode for translationally moving the surgical instrument 4 and a mode for rotationally moving the surgical instrument 4. In the mode for translationally moving the robot arm 60, the robot arm 60 is moved such that a distal end 4d of the surgical instrument 4 is moved in an X-Y plane. In the mode for rotationally moving the robot arm 60, the robot arm 60 is moved such that the surgical instrument 4 is rotationally moved around a center on the JT11 axis of the pair of forceps 4b or a distal end of the pair of forceps 4b as a fulcrum when any pivot position PP is not stored in a storage 32, and the surgical instrument 4 is rotationally moved around the pivot position PP as a fulcrum when the pivot position PP is stored in the storage 32. In such a case, the surgical instrument 4 is rotationally moved with the shaft 4c of the surgical instrument 4 inserted into a trocar T. The mode switching button 84 is arranged on a Z direction side surface of the arm operation unit 80.

The mode indicator 84a indicates a switched mode. The mode indicator 84a is on to indicate a rotational movement mode and is off to indicate a translational movement mode. Furthermore, the mode indicator 84a also serves as a pivot position indicator that indicates that the pivot position PP has been set. The mode indicator 84a is arranged on the Z direction side surface of the arm operation unit 80.

The pivot button 85 is a push-button switch to set the pivot position PP that serves as a fulcrum for movement of the surgical instrument 4 attached to the robot arm 60.

The adjustment button 86 is a button to optimize the position of the robot arm 60. After the pivot position PP for the robot arm 60 to which the endoscope 6 has been attached is set, the positions of the other robot arms 60 and the arm base 50 are optimized when the adjustment button 86 is pressed.

### Remote Control Apparatus

As shown in FIG. 1, the remote control apparatus 2 is arranged inside or outside the operating room, for example. The remote control apparatus 2 includes operation units 120 including arms 121 and operation handles 21, foot pedals 22, a touch panel 23, a monitor 24, a support arm 25, and a support bar 26. The operation units 120 include operation handles for the operator such as a doctor to input a command.

The operation units 120 each include a handle to operate the surgical instrument 4. The operation unit 120 receives an operation amount for the surgical instrument 4. The operation units 120 include an operation unit 120 that is located on the left side as viewed from the operator such as a doctor and is to be operated by the left hand of the operator, and an operation unit 120 that is located on the right side and is to be operated by the right hand of the operator. The operation unit 120 to be operated by the left hand of the operator and the operation unit 120 to be operated by the right hand of the operator include an operation handle 21L and an operation handle 21R, respectively.

The monitor 24 is a scope-type display that displays images captured by the endoscope 6. The support arm 25 supports the monitor 24 so as to align the height of the monitor 24 with the height of the face of the operator such as a doctor. The touch panel 23 is arranged on the support bar 26. The head of the operator is detected by a sensor provided in the vicinity of the monitor 24 such that the surgical robot 1 can be operated by the remote control apparatus 2. The operator operates the operation units 120 and the foot pedals 22 while visually recognizing an affected area on the monitor 24. Thus, a command is input to the remote control apparatus 2. The command input to the remote control apparatus 2 is transmitted to the surgical robot 1.

### Configuration of Medical Instrument, Adapter, Drape, and Arm

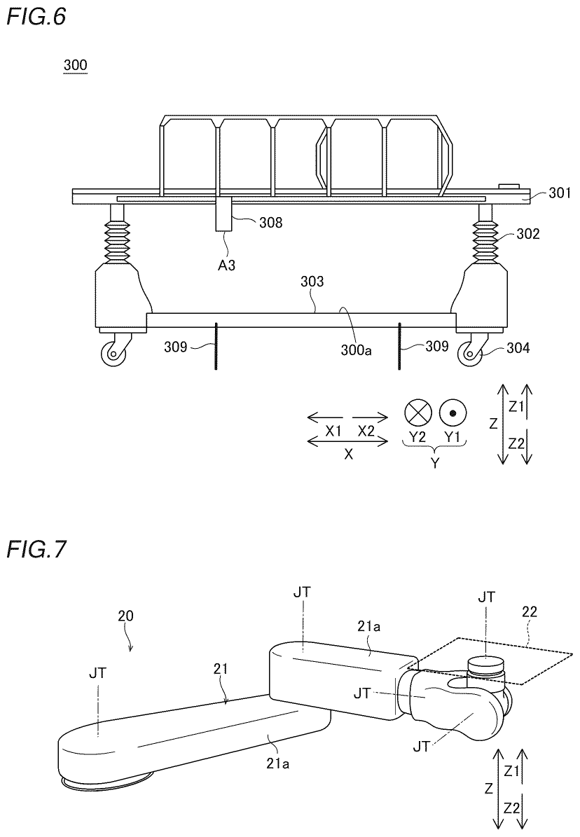

The configurations of the surgical instrument 4, an adapter 220, a drape 210, and the robot arm 60 are now described with reference to FIGS. 6 and 7.

As shown in FIGS. 6 and 7, the surgical instrument 4 is removably connected to the robot arm 60 via the adapter 220. The adapter 220 is arranged between a drive 75 of the robot arm 60 and the surgical instrument 4. The adapter 220 is a drape adapter for holding the drape 210, and is replaced by a user every time a surgery is performed. Thus, the drape 210 can be held using the adapter 220. The drape 210 is a drape for covering the robot arm 60 and is sterilized. The drape 210 is sandwiched between the adapter 220 and the robot arm 60. The drive 75 includes a first drive 751, a second drive 752, a third drive 753, and a fourth drive 754. The first drive 751 is an example of a drive.

The surgical instrument 4 includes a connector 4g that is an attachment surface arranged on the Y2 direction side, and the connector 4g is attached and connected to the adapter 220. The connector 4g is arranged in a housing 4h and is attached and connected to the robot arm 60 via the adapter 220. The adapter 220 includes a connector 220a that is an attachment surface arranged on the Y1 direction side, and the surgical instrument 4 is attached and connected to the connector 220a.

Furthermore, the adapter 220 includes a connector 220b that is an attachment surface arranged on the Y2 direction side, and the connector 220b is attached and connected to the drive 75 of the robot arm 60. The drive 75 of the robot arm 60 includes a connector 76 that is an attachment surface arranged on the Y1 direction side, and the adapter 220 is attached and connected to the connector 76.

As shown in FIG. 6, the robot arm 60 is covered with the drape 210 for use in a clean area. In the operating room, a cleaning operation is performed to prevent a surgically incised area and medical equipment from being contaminated with pathogens and foreign substances, for example. In this cleaning operation, the clean area and a contaminated area that is an area other than the clean area are set. The surgical site is placed in the clean area. Members of a surgical team including the operator must ensure that only sterile objects are placed in the clean area during surgery, and that an object placed in the contaminated area is sterilized when the object is moved to the clean area. Similarly, when the members of the surgical team including the operator place their hands in the contaminated area, they must sterilize their hands before making direct contact with objects located in the clean area. An instrument used in the clean area is sterilized or covered with the sterile drape 210.

The drape 210 includes a main body 211 that covers the robot arm 60, and a mount 212 that is sandwiched between the drive 75 of the robot arm 60 and the adapter 220. The main body 211 is made of a flexible film member formed into a film shape. The flexible film member is made of a resin material such as thermoplastic polyurethane or polyethylene. An opening is formed in the main body 211 such that the drive 75 of the robot arm 60 and the adapter 220 can engage with each other. The mount 212 is arranged at the opening of the main body 211 so as to close the opening. The mount 212 is made of a resin molded member. The resin molded member is made of a resin material such as polyethylene terephthalate. The mount 212 is harder and less likely to bend than the main body 211. The mount 212 includes an opening such that the drive 75 of the robot arm 60 and the adapter 220 can engage with each other. The mount 212 may include an opening to correspond to a portion in which the drive 75 of the robot arm 60 and the adapter 220 engage with each other. Alternatively, the mount 212 may include a plurality of openings to correspond to a plurality of portions in which the drive 75 of the robot arm 60 and the adapter 220 engage with each other.

As shown in FIGS. 6 and 7, the adapter 220 includes an adapter main body 221 and a plurality of drive transmitters 222 rotatably held around a rotation axis extending in the Y direction in the adapter main body 221. The plurality of drive transmitters 222 are arranged in the adapter main body 221 so as to be rotatable around the rotation axis. Four drive transmitters 222 are arranged so as to correspond to four driven members 4i of the surgical instrument 4. The drive transmitters 222 transmit a drive force from the robot arm 60 to the driven members 4i of the surgical instrument 4. The drive transmitters 222 include fitting recesses 222a into which fitting protrusions 4j of the driven members 41 of the surgical instrument 4 are fitted. The fitting recesses 222a are provided on the Y1 direction side, which is the surgical instrument 4 side of the drive transmitters 222, so as to be recessed from Y1 direction side surfaces of the drive transmitters 222 toward the Y2 direction side, which is the side opposite to the surgical instrument 4 side.

The drive transmitters 222 include fitting recesses 222b into which fitting protrusions 75a of the drive 75 of the robot arm 60 are fitted. The fitting recesses 222b are provided on the Y2 direction side, which is the robot arm 60 side of the drive transmitters 222, so as to be recessed from Y2 direction side surfaces of the drive transmitters 222 toward the Y1 direction side, which is the side opposite to the robot arm 60 side.

### Configuration of Control System

As shown in FIG. 8, the robotic surgical system 100 includes a control device 130, an arm controller 31a, a positioner controller 31b, and operation controllers 110.

The control device 130 is accommodated in the medical cart 3 to communicate with the arm controller 31a and the positioner controller 31b, and controls the entire robotic surgical system 100. Specifically, the control device 130 communicates with and controls the arm controller 31a, the positioner controller 31b, and the operation controllers 110. The control device 130 is connected to the arm controller 31a, the positioner controller 31b, and the operation controllers 110 through a LAN, for example. The control device 130 is placed inside the medical cart 3.

The arm controller 31a is arranged for each of the plurality of robot arms 60. That is, the same number of arm controllers 31a as the plurality of robot arms 60 are placed inside the medical cart 3.

As shown in FIG. 8, the input 33 is connected to the control device 130 through a LAN, for example. The status indicator 53, the arm status indicator 54, the operation handle 34, the throttle 34a, the joystick 33b, the stabilizer 34c, and the electric cylinder 34d are connected to the positioner controller 31b via a wire line 145 by means of a communication network that allows information to be shared with each other by using serial communication. Although FIG. 8 shows that the status indicator 53, the arm status indicator 54, etc. are all connected to one wire line 145, in reality, the wire line 145 is arranged for each of the status indicator 53, the arm status indicator 54, the operation handle 34, the throttle 34a, the joystick 33b, the stabilizer 34c, and the electric cylinder 34d.

As shown in FIG. 9, the arm portion 61 includes a plurality of servomotors M1, encoders E1, and speed reducers so as to correspond to a plurality of joints 64. The encoders E1 detect the rotation angles of the servomotors M1. The speed reducers slow down rotation of the servomotors M1 to increase the torques. Inside the medical cart 3, servo controllers C1 that control the Servomotors M1 are provided adjacent to the arm controller 31a. The encoders E1 that detect the rotation angles of the servomotors M1 are electrically connected to the servo controllers C1.

The joints 64 of the arm portion 61, and the joints 43 of the positioner 40 include brakes BRK. Furthermore, the front wheels of the medical cart 3, the arm base 50, and the translation mechanism 70 include brakes BRK. The arm controller 31a unidirectionally transmits control signals to the brakes BRK of the joints 64 of the arm portion 61 and the translation mechanism 70. The control signals are signals for turning on/off the brakes BRK.

The signals for turning on the brakes BRK include signals for maintaining the brakes BRK in an enabled state. The same applies to control signals from the positioner controller 31b to the brakes BRK of the joints 43 of the positioner 40 and the arm base 50. On startup, all the brakes BRK of the arm base 50, the arm portion 61, and the translation mechanism 70 are turned off but servomotors SM are driven against gravity to maintain the postures of the robot arm 60 and the arm base 50. When an error occurs in the robotic surgical system 100, the brakes BRK of the arm base 50, the arm portion 61 and the translation mechanism 70 are turned on. When the error in the robotic surgical system 100 is reset, the brakes BRK of the arm base 50, the arm portion 61, and the translation mechanism 70 are turned off. When a shutdown operation is performed in the robotic surgical system 100, the brakes BRK of the arm base 50, the arm portion 61, and the translation mechanism 70 are turned on. The brakes BRK of the front wheels of the medical cart 3 are constantly turned on, and the brakes BRK are deactivated only while the enable switch 34b of the cart positioner operation unit 35 is being pressed. The brakes BRK of the joints 43 of the positioner 40 are constantly turned on, and the brakes BRK are deactivated only while the enable switch 33c of the cart positioner operation unit 35 is being pressed.

Servomotors M2 to rotate the driven members provided in the driven unit 4a of the surgical instrument 4, encoders E2, and speed reducers are arranged in the second link 73. The encoders E2 detect the rotation angles of the servomotors M2. The speed reducers slow down rotation of the servomotors M2 to increase the torques. In the medical cart 3, servo controllers C2 are provided to control the servomotors M2 to drive the surgical instrument 4. The encoders E2 that detect the rotation angles of the servomotors M2 are electrically connected to the servo controllers C2. A plurality of servomotors M2, a plurality of encoders E2, and a plurality of servo controllers C2 are arranged.

The translation mechanism 70 includes a servomotor M3 to translationally move the surgical instrument 4, an encoder E3, and a speed reducer. The encoder E3 detects the rotation angle of the servomotor M3. The speed reducer slows down rotation of the servomotor M3 to increase the torque. In the medical cart 3, a servo controller C3 is provided to control the servomotor M3 to translationally move the surgical instrument 4. The encoder E3 that detects the rotation angle of the servomotor M3 is electrically connected to the servo controller C3.

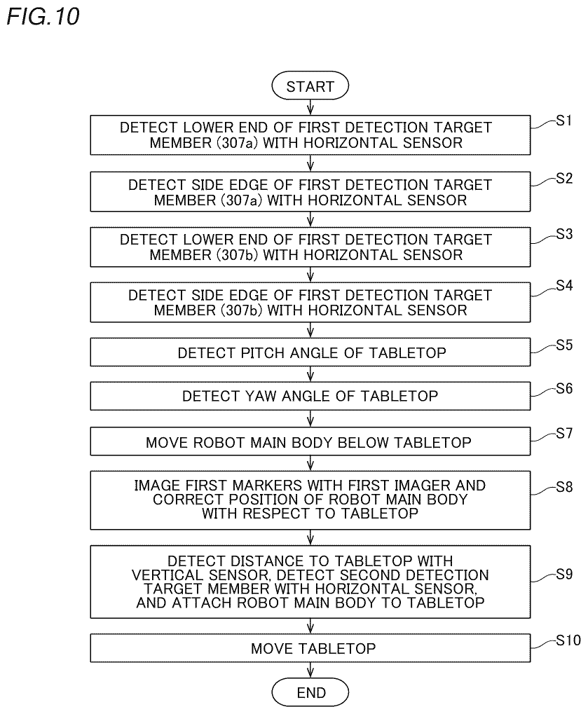

As shown in FIG. 10, the positioner 40 includes a plurality of servomotors M4, encoders E4, and speed reducers so as to correspond to a plurality of joints 43 of the positioner 40. The encoders E4 detect the rotation angles of the servomotors M4. The speed reducers slow down rotation of the servomotors M4 to increase the torques.

The medical cart 3 includes wheels including front wheels as drive wheels and rear wheels that are steered by the operation handle 34. The rear wheels are arranged closer to the operation handle 34 than the front wheels.

The medical cart 3 includes servomotors M5 to drive a plurality of front wheels of the medical cart 3, encoders E5, speed reducers, and brakes. The speed reducers slow down rotation of the servomotors M5 to increase the torques. A potentiometer P1 shown in FIG. 2 is provided on the operation handle 34 of the cart positioner operation unit 35, and the servomotors M5 of the front wheels are driven based on a rotation angle detected by the potentiometer Pl according to the twist of the throttle 34a. Rear wheels of the medical cart 3 are of the dual wheel type, and the rear wheels are steered based on rightward-leftward rotation of the operation handle 34. Furthermore, a potentiometer P2 shown in FIG. 2 is provided on a rotation axis of the operation handle 34 of the cart positioner operation unit 35, and servomotors M5a, encoders E5a, and speed reducers are provided on the rear wheels of the medical cart 3. The speed reducers slow down rotation of the servomotors M5a to increase the torques. The servomotors M5a are driven based on a rotation angle detected by the potentiometer P2 according to rightward-leftward rotation of the operation handle 34. That is, steering of the rear wheels by the rightward-leftward rotation of the operation handle 34 is power-assisted by the servomotors M5a.

The front wheels of the medical cart 3 are driven such that the medical cart 3 moves in a forward-rearward direction. Furthermore, the operation handle 34 of the cart positioner operation unit 35 is rotated such that the rear wheels are steered, and the medical cart 3 turns in a right-left direction.

As shown in FIG. 10, in the medical cart 3, servo controllers C4 are provided to control the servomotors M4 to move the positioner 40. The encoders E4 that detect the rotation angles of the servomotors M4 are electrically connected to the servo controllers C4. In the medical cart 3, servo controllers C5 are provided to control the Servomotors M5 to drive the front wheels of the medical cart 3. The encoders E5 that detect the rotation angles of the servomotors M5 are electrically connected to the Servo controllers C5. In the medical cart 3, servo controllers C5a are provided to control the servomotors M5a to power-assist steering of the rear wheels of the medical cart 3. The encoders E5a that detect the rotation angles of the servomotors M5a are electrically connected to the servo controllers C5a.

As shown in FIG. 8, the control device 130 controls the robot arm 60 based on an operation received by the arm operation unit 80. For example, the control device 130 controls the robot arm 60 based on an operation received by the joystick 82 of the arm operation unit 80.

Specifically, the arm controller 31a outputs an input signal input from the joystick 82 to the control device 130. The control device 130 generates position commands based on the received input signal and the rotation angles detected by the encoders E1, and outputs the position commands to the servo controllers C1 via the arm controller 31a. The servo controllers C1 generate current commands based on the position commands input from the arm controller 31a and the rotation angles detected by the encoders E1, and output the current commands to the Servomotors M1. Thus, the robot arm 60 is moved according to an operation command input to the joystick 82.

The control device 130 controls the robot arm 60 based on an input signal from either linear switch 83 of the arm operation unit 80. Specifically, the arm controller 31a outputs the input signal input from the linear switch 83 to the control device 130. The control device 130 generates a position command(s) based on the received input signal and the rotation angle(s) detected by the encoders E1 or the encoder E3, and outputs the position command(s) to the servo controllers C1 or the servo controller C3 via the arm controller 31a. The servo controllers C1 or the servo controller C3 generates a current command(s) based on the position command(s) input from the arm controller 31a and the rotation angle(s) detected by the encoders E1 or the encoder E3, and outputs the current command(s) to the servomotors M1 or the servomotor M3. Thus, the robot arm 60 is moved according to an operation command input to the linear switch 83.

The positioner controller 31b is arranged in the medical cart 3. The positioner controller 31b controls the positioner 40 and the medical cart 3. The servomotors SM, the encoders EN, and the speed reducers are provided in the positioner 40 so as to correspond to the plurality of joints 43 of the positioner 40. The servo controllers SC are provided in the medical cart 3 to control the Servomotors SM of the positioner 40. The servomotors SM that drive the plurality of front wheels of the medical cart 3, the encoders EN, the speed reducers, the servo controllers SC, and the brakes are provided in the medical cart 3.

The operation controllers 110 are arranged in a main body of the remote control apparatus 2. The operation controllers 110 control the operation units 120. The operation controllers 110 are provided so as to correspond to the left-handed operation unit 120 and the right-handed operation unit 120, respectively. Servomotors SM, encoders EN, and speed reducers are provided in the operation units 120 so as to correspond to a plurality of joints of the operation units 120. Servo controllers SC that control the servomotors SM of the operation units 120 are provided adjacent to the operation controllers 110 in the main body of the remote control apparatus 2.

### Configuration of Manual Surgical Instrument

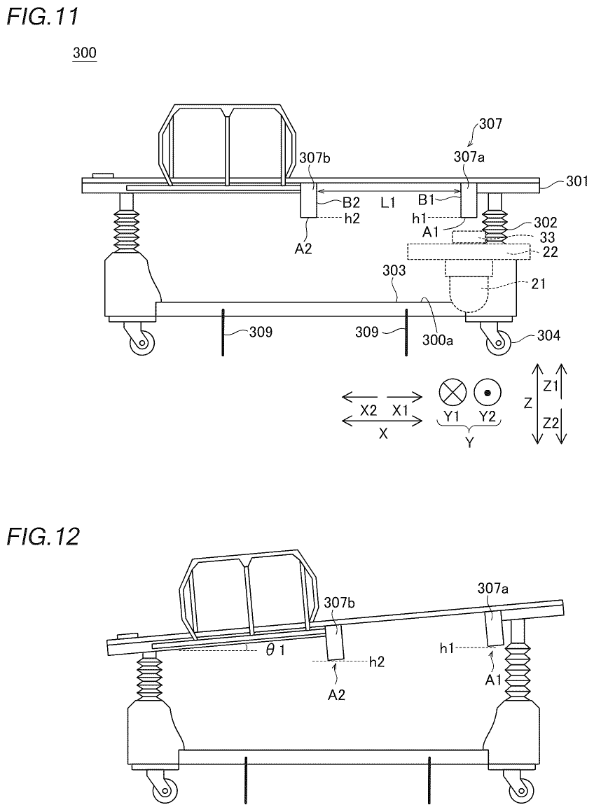

The configuration of a manual surgical instrument 400 is now described with reference to FIG. 11. Although the manual surgical instrument 400 is originally an instrument to be operated by the operator such as a doctor, in this embodiment, it is not directly operated by a doctor, but is operated by the surgical robot 1 via the remote control apparatus 2.

The manual surgical instrument 400 includes a grip 401, an operation handle 402, a rotating portion 403, a shaft 404, and an end effector 405. The end effector 405 includes a pair of jaw members arranged at a distal end of the shaft 404.

The operation handle 402 has an annular shape with a hole disposed in the center. The operator such as a doctor grasps the grip 401 and the operation handle 402. Thus, the grip 401 contacts the palm of the operator's hand. The operator hooks his/her fingers on the inside of the annular operation handle 402. When the operator grasps the operation handle 402, the operation handle 402 rotates in an A1 direction. This causes the pair of jaw members to close. When the operator releases his/her grip of the operation handle 402, the operation handle 402 rotates in an A2 direction. This causes the pair of jaw members to open.

The rotating portion 403 is connected to the shaft 404. When the rotating portion 403 rotates in a B1 direction or a B2 direction, the shaft 404 rotates in the B1 direction or the B2 direction.

### Configuration of Surgical Instrument Adapter

The configuration of a surgical instrument adapter 300 is now described. The surgical instrument adapter 300 is used to connect the manual surgical instrument 400 to the robot arm 60.

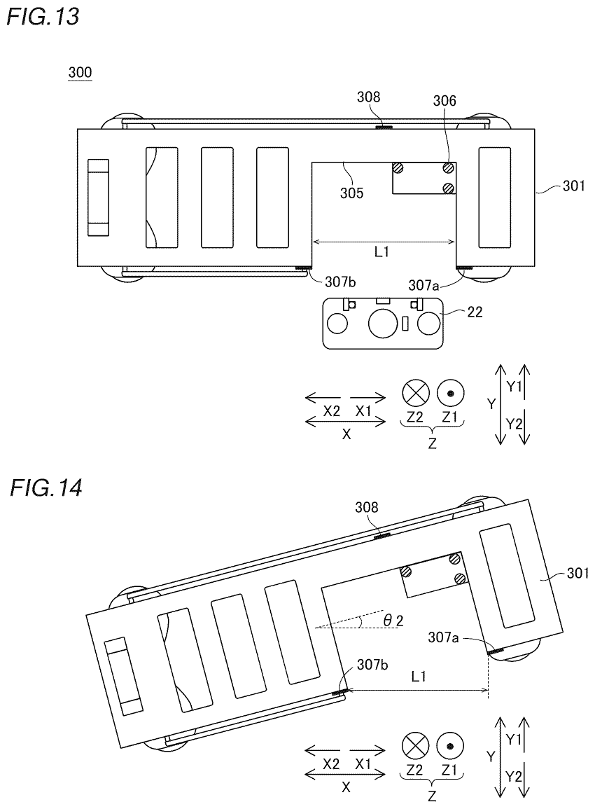

In this embodiment, the surgical instrument adapter 300 includes an interface 310 shown in FIG. 12, a first driven portion 320, a first drive force transmission mechanism 330, a second pulley 340, and a surgical instrument holder 350 shown in FIG. 13, and a second driven portion 360 and a second drive force transmission mechanism 370 shown in FIG. 18. The first driven portion 320 is an example of a driven portion. The first drive force transmission mechanism 330 is an example of a drive force transmission mechanism.

As shown in FIG. 14, the interface 310 includes a first rotor 311 to be rotationally driven by a first drive force transmitted from the first drive 751 arranged in the robot arm 60 shown in FIG. 6. The interface 310 includes a second rotor 312 to be rotationally driven by a second drive force transmitted from a second drive 752 arranged in the robot arm 60. The interface 310 is attached to the holder 71 of the second link 73 shown in FIG. 6 via the adapter 220. The first rotor 311 and the second rotor 312 are pulleys. A pulley is also called a capstan. A fitting protrusion 311a and a fitting protrusion 312a are arranged on the second link 73 side of the first rotor 311 and the second rotor 312, respectively. The fitting protrusion 311a and the fitting protrusion 312a are fitted into the fitting recesses 222a of the adapter 220 shown in FIG. 6. The first rotor 311 and the second rotor 312 rotate around an axis F1 and an axis F2, respectively. The axis F1 and the axis F2 are along the Y direction. The first rotor 311 is an example of a rotor.

In this embodiment, as shown in FIG. 13, the first driven portion 320 rotates the operation handle 402 of the manual surgical instrument 400 with the first drive force transmitted from the first drive 751. The first driven portion 320 rotates the operation handle 402 in the A1 direction and the A2 direction.

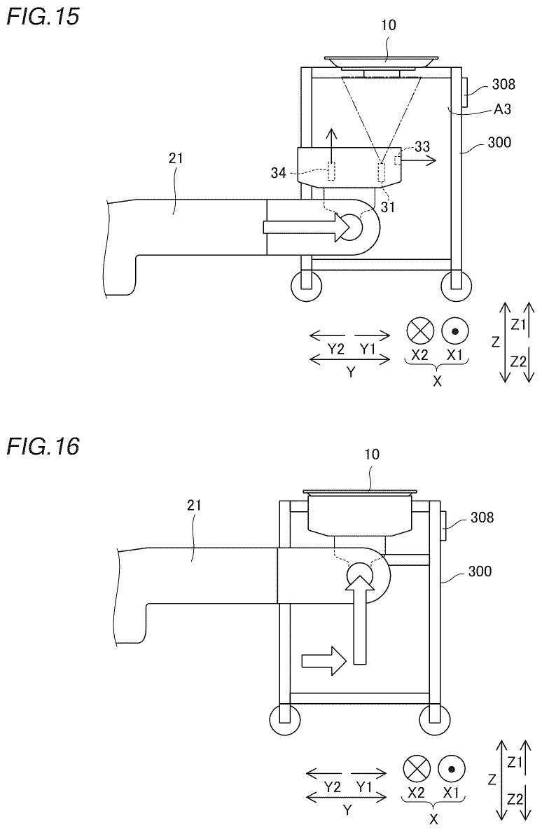

In this embodiment, the first driven portion 320 includes a lever 321, a roller 322 shown in FIG. 15, a sandwiching portion 323, and a first pulley 324. The lever 321 is rotated by the first drive force.

Specifically, the lever 321 is fixed to the inner surface of the surgical instrument holder 350. The lever 321 rotates around an axis F3 as a rotation axis. The axis F3 is along the X direction. The lever 321 has a plate shape.

In this embodiment, as shown in FIG. 15, the roller 322 is connected to the lever 321 and contacts the operation handle 402. The roller 322 moves while rotating along a surface 402a of the operation handle 402. The surface 402a is a surface of the operation handle 402 on the Z1 side. The roller 322 is made of a metal, for example. The roller 322 is connected to the distal end side, which is a first side of the lever 321. The roller 322 is rotatably connected to the distal end side of the lever 321. The roller 322 is connected to the lever 321 in a cantilever shape. The roller 322 rotates around an axis F4 as a rotation axis. The axis F4 is along the X direction. The roller 322 has a cylindrical shape. The roller 322 is in line contact with the operation handle 402.

In this embodiment, the sandwiching portion 323 sandwiches the operation handle 402 with the roller 322.

The sandwiching portion 323 contacts the inner surface 402b of the annular operation handle 402. The surface 402b is a surface on the Z2 side opposite the surface 402a on the Z1 side.

In this embodiment, as shown in FIG. 16, the sandwiching portion 323 includes a main body 323a, a contact portion 323b, and an urging portion 323c. The main body 323a is attached to the lever 321. The main body 323a has an L-shape. The proximal end side of the main body 323a is attached to the distal end side of the lever 321. A groove 323d is arranged on the proximal end side of the main body 323a, and the lever 321 is inserted into the groove 323d. The main body 323a does not rotate with respect to the lever 321. The main body 323a is attached to the lever 321 by a fastening member such as a screw.

The contact portion 323b contacts the operation handle 402. The contact portion 323b contacts the inner surface 402b of the annular operation handle 402. In this embodiment, a portion 323e of the contact portion 323b that contacts the operation handle 402 includes a curved surface. The contact portion 323b has a substantially semi-cylindrical shape. The portion 323e of the contact portion 323b on the Z1 side includes a curved surface, and a portion 323f of the contact portion 323b on the Z2 side includes a flat surface.

In this embodiment, the urging portion 323c is arranged between the main body 323a and the contact portion 323b. The urging portion 323c urges the contact portion 323b toward the operation handle 402. The contact portion 323b is a compression coil spring, for example. A first end of the urging portion 323c is connected to the portion 323f of the contact portion 323b. A second end of the urging portion 323c is connected to the main body 323a. A groove 323g is arranged in the main body 323a.

The groove 323g has a cross shape. The groove 323g is formed along the Z direction and also formed along the X direction. A second end of the urging portion 323c is connected to the inner surface of the groove 323g on the Z2 side. The urging portion 323c and the contact portion 323b are arranged in a portion of the groove 323g formed along the Z direction.

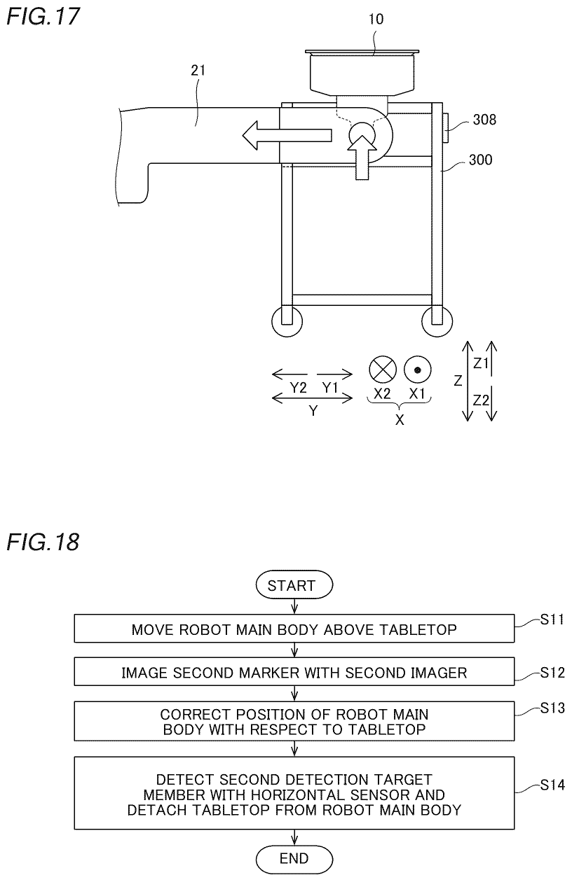

In this embodiment, as shown in FIG. 17, the first pulley 324 is connected to the proximal end side, which is a second side of the lever 321. The first pulley 324 is rotated by a second elongate element 332 described below.

In this embodiment, the first drive force transmission mechanism 330 transmits the first drive force from the first rotor 311 to the first driven portion 320. The first drive force transmission mechanism 330 is arranged between the first rotor 311 and the first driven portion 320. The first drive force transmission mechanism 330 includes an elongate element including a wire or a cable that transmits the first drive force from the first rotor 311 to the first driven portion 320. The first drive force transmission mechanism 330 includes a first elongate element 331 wound around the first rotor 311 and the second pulley 340, and the second elongate element 332 wound around the second pulley 340 and the first pulley 324 of the first driven portion 320. The first elongate element 331 and the second elongate element 332 are examples of an elongate element.

In this embodiment, the second pulley 340 is arranged in a movement path of the elongate element between the first rotor 311 and the first pulley 324. As described above, the first elongate element 331 is wound around the first rotor 311 and the second pulley 340. The second elongate element 332 is wound around the second pulley 340 and the first pulley 324. In the second pulley 340, the first elongate element 331 and the second elongate element 332 are offset in the X direction. In the surgical instrument holder 350, the second pulley 340 is arranged on the Z2 direction side of the first rotor 311. The second pulley 340 is arranged on the interface 310. The first elongate element 331 is arranged along the Z direction. The second elongate element 332 is disposed so as to intersect with the Z direction. The second pulley 340 rotates around an axis F5 as a rotation axis. The axis F5 is along the X direction.

In this embodiment, as shown in FIG. 13, the diameter d1 of the first pulley 324 is larger than the diameter d2 of the first rotor 311. The diameters d1 and d2 are set such that the first driven portion 320 can rotate the operation handle 402 by the drive force of the first drive 751. The diameter d2 of the first rotor 311 indicates the diameter of a shaft around which the first elongate element 331 is wound.

In this embodiment, the surgical instrument holder 350 holds the manual surgical instrument 400 so as to position the roller 322 with respect to the operation handle 402. The surgical instrument holder 350 is a housing that covers the grip 401, the operation handle 402, and the rotating portion 403 of the manual surgical instrument 400. As shown in FIG. 12, the surgical instrument holder 350 includes a main body 351 and a lid 352. The lid 352 is attached to the main body 351 by a hinge 353. The lid 352 covers an opening of the main body 351. The main body 351 includes a hole 354. The shaft 404 of the manual surgical instrument 400 is inserted into the hole 354, and the rotating portion 403 is positioned in the hole 354. As shown in FIG. 13, the grip 401 and the operation handle 402 are arranged in the main body 351. The operation handle 402 is disposed in the main body 351 so as to contact the roller 322.

In this embodiment, the interface 310 and the first driven portion 320 are arranged in the surgical instrument holder 350. The interface 310 is arranged on the Y2 side of the main body 351 of the surgical instrument holder 350. The first driven portion 320 is attached to the inner surface of the main body 351 on the X2 side. The annular operation handle 402 is disposed in the main body 351 such that t the sandwiching portion 323 is inserted into a central hole of the annular operation handle 402.

In this embodiment, as shown in FIG. 18, the second driven portion 360 rotates the rotating portion 403 of the manual surgical instrument 400. The second drive force transmission mechanism 370 transmits the second drive force from the second rotor 312 to the second driven portion 360. The second drive force transmission mechanism 370 includes a gearing 371 that is arranged between the second rotor 312 and the second driven portion 360 and transmits the second drive force from the second rotor 312 to the second driven portion 360. A helical gear 312b is arranged on the second rotor 312. The gearing 371 includes a helical gear 371a and a spur gear 371b. The second driven portion 360 includes a cylindrical member 361 and a spur gear 362. The rotating portion 403 of the manual surgical instrument 400 is inserted into the cylindrical member 361. The cylindrical member 361 is arranged in the hole 354 of the surgical instrument holder 350. The spur gear 362 is arranged on the outer periphery of the cylindrical member 361. The helical gear 312b of the second rotor 312 meshes with the helical gear 371a. The spur gear 371b meshes with the spur gear 362. Thus, the second drive force is transmitted from the second drive 752 to the second driven portion 360.

As shown in FIG. 13, a slip-out prevention member 356 that prevents the rotating portion 403 of the manual surgical instrument 400 from slipping out of the cylindrical member 361 in a Z2 direction is arranged on the inner surface of the surgical instrument holder 350.

The slip-out prevention member 356 contacts the grip 401 such that the rotating portion 403 of the manual surgical instrument 400 is prevented from slipping out of the cylindrical member 361 in the Z2 direction.

### Operation

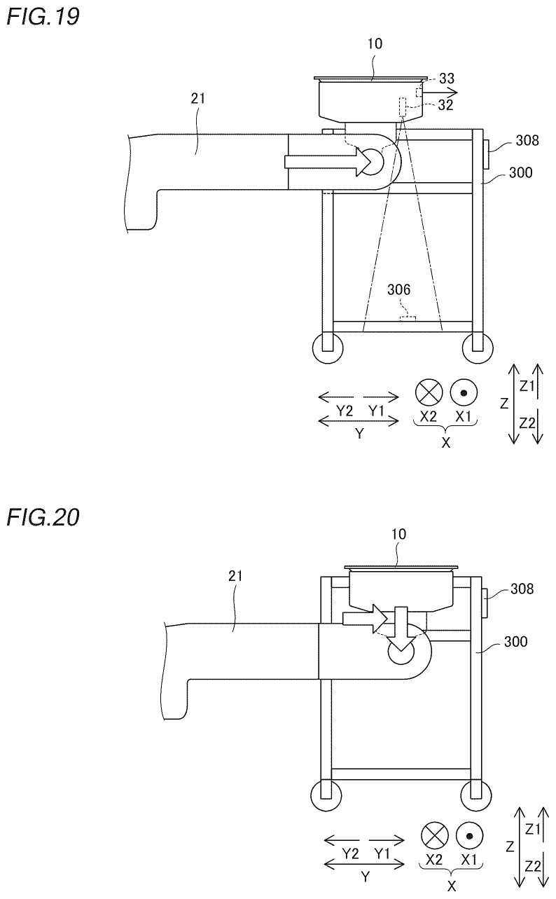

As shown in FIG. 19, the first rotor 311 is rotated around the axis F1 in a G1 direction by the first drive force transmitted from the first drive 751 such that the first elongate element 331 rotates the second pulley 340 around the axis F5 in a G2 direction. The rotation of the second pulley 340 causes the second elongate element 332 to rotate the first pulley 324 around the axis F3 in a G3 direction. Thus, the roller 322 moves along the surface 402a of the operation handle 402 such that the operation handle 402 rotates in the A1 direction. As shown in FIG. 13, the first rotor 311 rotates around the axis F1 in a direction opposite to the G1 direction such that the contact portion 323b moves along the surface 402b of the operation handle 402, and the operation handle 402 rotates in the A2 direction.

As shown in FIG. 18, the second rotor 312 is rotated around the axis F2 in a G11 direction by the second drive force transmitted from the second drive 752 such that the helical gear 371a and the spur gear 371b of the gearing 371 rotate in a G12 direction. The spur gear 371b of the gearing 371 rotates in the G12 direction such that the spur gear 362 rotates in a G13 direction. The spur gear 362 and the cylindrical member 361 rotate such that the rotating portion 403 of the manual surgical instrument 400 rotates in the G13 direction. The second rotor 312 rotates around the axis F2 in a direction opposite to the G11 direction such that the rotating portion 403 of the manual surgical instrument 400 rotates in a direction opposite to the G13 direction.

### Advantages of This Embodiment

The first driven portion 320 includes the lever 321 to be rotated by the first drive force, and the roller 322 connected to the lever 321, contacting the operation handle 402, and operable to move while rotating along the surface 402 of the operation handle 402. Accordingly, the roller 322 of the first driven portion 320 moves while rotating along the surface 402a of the operation handle 402, and thus a frictional force acting between the roller 322 and the operation handle 402 is relatively small.

Therefore, the first driven portion 320 can smoothly rotate the operation handle 402 of the manual surgical instrument 400.

The first driven portion 320 further includes the sandwiching portion 323 to sandwich the operation handle 402 with the roller 322. Accordingly, the roller 322 can rotate the operation handle 402 in the Al direction, and the sandwiching portion 323 allows the operation handle 402 to rotate in the A2 direction opposite to the Al direction. That is, the rotation of the operation handle 402 in both directions can be controlled by the first drive force of the first drive 751. In addition, the sandwiching portion 323 can reduce or prevent spacing apart of the roller 322 from the operation handle 402.

The sandwiching portion 323 includes the main body 323a attached to the lever 321, the contact portion 323b contacting the operation handle 402, and the urging portion 323c between the main body 323a and the contact portion 323b to urge the contact portion 323b toward the operation handle 402. Accordingly, the urging portion 323c urges the contact portion 323b toward the operation handle 402, and thus the contact between the operation handle 402 and the contact portion 323b can be maintained even when a distance between the operation handle 402 and the contact portion 323b changes as the operation handle 402 rotates.

The portion 323e of the contact portion 323b contacting the operation handle 402 includes the curved surface. Accordingly, the frictional force acting between the contact portion 323b of the sandwiching portion 323 and the operation handle 402 is relatively small, and thus the sandwiching portion 323 can be moved smoothly with respect to the operation handle 402.

The first drive force transmission mechanism 330 includes the first elongate element 331 and the second elongate element 332 between the first rotor 311 and the first driven portion 320 including the wire or the cable to transmit the first drive force from the first rotor 311 to the first driven portion 320. The roller 322 is connected to the first side of the lever 321, and the first driven portion 320 further includes the first pulley 324 connected to the second side of the lever 321 and to be rotated by the second elongate element 332. Accordingly, the first elongate element 331 and the second elongate element 332 are exposed so as to be easily cleaned.

The surgical instrument adapter 300 further includes the second pulley 340 in the movement paths of the first elongate element 331 and the second elongate element 332 between the first rotor 311 and the first pulley 324. Accordingly, even when a component that interferes with the first elongate element 331 and the second elongate element 332 is placed between the first rotor 311 and the first pulley 324, the second pulley 340 can be used to change the movement paths of the first elongate element 331 and the second elongate element 332 such that interference between the component and both the first elongate element 331 and the second elongate element 332 can be reduced or prevented.

The diameter d1 of the first pulley 324 is larger than the diameter d2 of the first rotor 311. Accordingly, the torque of the first drive 751 can be transmitted to the first driven portion 320 in an increased state via the first rotor 311, the second pulley 340, and the first pulley 324. Therefore, a drive force for rotating the operation handle 402 can be ensured without increasing the size of the first drive 751.

The surgical instrument adapter 300 further includes the surgical instrument holder 350 to hold the manual surgical instrument 400 so as to position the roller 322 with respect to the operation handle 402. Accordingly, the manual surgical instrument 400 is held by the surgical instrument holder 350 such that the roller 322 can be easily positioned with respect to the operation handle 402.

The cylindrical member 361 is arranged in the surgical instrument holder 350 and is connected to the interface 310 via a bearing. The rotating portion 403 of the manual surgical instrument 400 engages with the cylindrical member 361 such that the manual surgical instrument 400 can be easily positioned. Specifically, the rotating portion 403 connected to the shaft 404 of the manual surgical instrument 400 includes a notch. The cylindrical member 361 of the surgical instrument holder 350 is connected so as to fit into the notch of the rotating portion 403 such that the manual surgical instrument 400 is positioned with respect to the surgical instrument holder 350.

The interface 310 further includes the second rotor 312 to be rotationally driven by the second drive force transmitted from the second drive 752 in the robot arm 60. The surgical instrument adapter 300 further includes the second driven portion 360 to rotate the rotating portion 403 connected to the shaft 404 of the manual surgical instrument 400, and the second drive force transmission mechanism 370 to transmit the second drive force from the second rotor 312 to the second driven portion 360. Accordingly, the rotating portion 403 of the manual surgical instrument 400 can be easily rotated by the second drive force of the second drive 752.

The second drive force transmission mechanism 370 includes the gearing 371 between the second rotor 312 and the second driven portion 360 to transmit the second drive force from the second rotor 312 to the second driven portion 360. Accordingly, the gearing 371 having a relatively simple configuration can easily rotate the rotating portion 403 of the manual surgical instrument 400 with the second drive force of the second drive 752.

### Modified Examples

The embodiment disclosed this time must be considered as illustrative in all points and not restrictive. The scope of the present disclosure is not shown by the above description of the embodiment but by the scope of claims for patent, and all modifications (modified examples) within the meaning and scope equivalent to the scope of claims for patent are further included.

While the example in which the first drive force transmission mechanism 330 includes the first elongate element 331 and the second elongate element 332 including the wire or the cable has been shown in the aforementioned embodiment, the present disclosure is not limited to this. For example, the first drive force transmission mechanism 330 may include a guide wire.

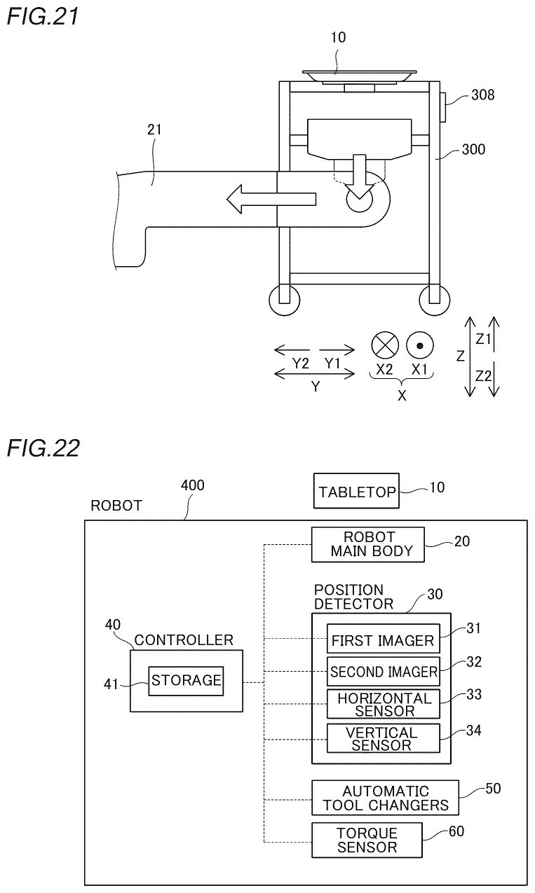

While the example in which the first driven portion 320 includes the sandwiching portion 323 has been shown in the aforementioned embodiment, the present disclosure is not limited to this. For example, as shown in a modified example of FIG. 20, a first driven portion 420 may not include a sandwiching portion 323. The first driven portion 420 includes only a roller 322 connected to a lever 321. The first driven portion 420 is an example of a drive.

While the example in which the contact portion 323b of the sandwiching portion 323 has a substantially semi-cylindrical shape has been shown in the aforementioned embodiment, the present disclosure is not limited to this. For example, the contact portion may include a cylindrical and rotatable roller.

While the example in which both the first driven portion 320 that rotates the operation handle 402 of the manual surgical instrument 400 and the second driven portion 360 that rotates the rotating portion 403 of the manual surgical instrument 400 are arranged in the surgical instrument adapter 300 has been shown in the aforementioned embodiment, the present disclosure is not limited to this. For example, only the first driven portion 320 that rotates the operation handle 402 of the manual surgical instrument 400 may be arranged in the surgical instrument adapter 300.

While the example in which four robot arms 60 are provided has been shown in the aforementioned embodiment, the present disclosure is not limited to this. In the present disclosure, the number of robot arms 60 may be any number as long as at least one robot arm 60 is provided.

While the example in which each of the arm portion 61 and the positioner 40 includes a 7-axis articulated robot has been shown in the aforementioned embodiment, the present disclosure is not limited to this. For example, each of the arm portion 61 and the positioner 40 may include an articulated robot having an axis configuration other than the 7-axis articulated robot. The axis configuration other than the 7-axis articulated robot includes six axes or eight axes, for example.

While the example in which the surgical robot 1 includes the medical cart 3, the positioner 40, and the arm base 50 has been shown in the aforementioned embodiment, the present disclosure is not limited to this. For example, the surgical robot 1 may not include the medical cart 3, the positioner 40, or the arm base 50, but may include only the robot arms 60.

### ASPECTS

It will be appreciated by those skilled in the art that the exemplary embodiments described above are specific examples of the following aspects.

### Item 1

A surgical instrument adapter operable to connect a manual surgical instrument to a robot arm, the surgical instrument adapter comprising:
an interface including a first rotor to be rotationally driven by a first drive force transmitted from a first drive in the robot arm;
a first driven portion to rotate an operation handle of the manual surgical instrument; and
a first drive force transmission mechanism to transmit the first drive force from the first rotor to the first driven portion; wherein
the first driven portion includes:
   a lever to be rotated by the first drive force; and
   a roller connected to the lever, contacting the operation handle, and operable to move while rotating along a surface of the operation handle.

### Item 2

The surgical instrument adapter according to item 1, wherein the first driven portion further includes a sandwiching portion to sandwich the operation handle with the roller.

### Item 3

The surgical instrument adapter according to item 2, wherein the sandwiching portion includes:
a main body attached to the lever;
a contact portion contacting the operation handle; and
an urging portion between the main body and the contact portion to urge the contact portion toward the operation handle.

### Item 4

The surgical instrument adapter according to item 3, wherein a portion of the contact portion contacting the operation handle includes a curved surface.

### Item 5

The surgical instrument adapter according to any one of items 1 to 4, wherein
the first drive force transmission mechanism includes an elongate element between the first rotor and the first driven portion including a wire or a cable to transmit the first drive force from the first rotor to the first driven portion;
the roller is connected to a first side of the lever; and
the first driven portion further includes a first pulley connected to a second side of the lever and to be rotated by the elongate element.

### Item 6

The surgical instrument adapter according to item 5, wherein the first pulley has a diameter larger than a diameter of the first rotor.

### Item 7

The surgical instrument adapter according to item 5, further comprising:
a second pulley in a movement path of the elongate element between the first rotor and the first pulley.

### Item 8

The surgical instrument adapter according to any one of items 1 to 7, further comprising:
a surgical instrument holder to hold the manual surgical instrument so as to position the roller with respect to the operation handle.

### Item 9

The surgical instrument adapter according to item 8, wherein the interface and the first driven portion are arranged in the surgical instrument holder.

### Item 10

The surgical instrument adapter according to any one of items 1 to 9, wherein
the interface further includes a second rotor to be rotationally driven by a second drive force transmitted from a second drive in the robot arm; and
the surgical instrument adapter further comprises:
   a second driven portion to rotate a rotating portion connected to a shaft of the manual surgical instrument; and
   a second drive force transmission mechanism to transmit the second drive force from the second rotor to the second driven portion.

### Item 11

The surgical instrument adapter according to item 10, wherein the second drive force transmission mechanism includes a gearing between the second rotor and the second driven portion to transmit the second drive force from the second rotor to the second driven portion.

### Item 12

A surgical robot comprising:
a robot arm including a drive;
a surgical instrument adapter to operably connect a manual surgical instrument to the robot arm; and
an interface including a rotor to be rotationally driven by a drive force transmitted from the drive in the robot arm; wherein
the surgical instrument adapter includes:
   a driven portion to rotate an operation handle of the manual surgical instrument; and
   a drive force transmission mechanism to transmit the drive force from the rotor to the driven portion; and the driven portion includes:
   a lever to be rotated by the drive force; and
   a roller connected to the lever, contacting the operation handle, and operable to move along a surface of the operation handle.
60: robot arm
100: surgical robot
300: surgical instrument adapter
310: interface
311: first rotor (rotor)
312: second rotor
320, 420: first driven portion (driven portion)
321: lever
322: roller
323: sandwiching portion
323a: main body
323b: contact portion
323c: urging portion
323e: portion
324: first pulley
330: first drive force transmission mechanism (drive force transmission mechanism)
331: first elongate element (elongate element)
332: second elongate element (elongate element)
340: second pulley
350: surgical instrument holder
360: second driven portion
370: second drive force transmission mechanism
371: gearing
400: manual surgical instrument
402: operation handle
403: rotating portion
404: shaft
751: first drive (drive)
752: second drive

1. A surgical instrument adapter operable to connect a manual surgical instrument to a robot arm, the surgical instrument adapter comprising:
an interface including a first rotor to be rotationally driven by a first drive force transmitted from a first drive in the robot arm;
a first driven portion to rotate an operation handle of the manual surgical instrument; and
a first drive force transmission mechanism to transmit the first drive force from the first rotor to the first driven portion; wherein
the first driven portion includes:
a lever to be rotated by the first drive force; and
a roller connected to the lever, contacting the operation handle, and operable to move while rotating along a surface of the operation handle.

2. The surgical instrument adapter according to claim 1, wherein the first driven portion further includes a sandwiching portion to sandwich the operation handle with the roller.

3. The surgical instrument adapter according to claim 2, wherein the sandwiching portion includes:
a main body attached to the lever;
a contact portion contacting the operation handle; and
an urging portion between the main body and the contact portion to urge the contact portion toward the operation handle.

4. The surgical instrument adapter according to claim 3, wherein a portion of the contact portion contacting the operation handle includes a curved surface.

5. The surgical instrument adapter according to claim 1, wherein
the first drive force transmission mechanism includes an elongate element between the first rotor and the first driven portion including a wire or a cable to transmit the first drive force from the first rotor to the first driven portion;
the roller is connected to a first side of the lever; and
the first driven portion further includes a first pulley connected to a second side of the lever and to be rotated by the elongate element.

6. The surgical instrument adapter according to claim 5, wherein the first pulley has a diameter larger than a diameter of the first rotor.

7. The surgical instrument adapter according to claim 5, further comprising:
a second pulley in a movement path of the elongate element between the first rotor and the first pulley.

8. The surgical instrument adapter according to claim 1, further comprising:
a surgical instrument holder to hold the manual surgical instrument so as to position the roller with respect to the operation handle.

9. The surgical instrument adapter according to claim 8, wherein the interface and the first driven portion are arranged in the surgical instrument holder.

10. The surgical instrument adapter according to claim 1, wherein
the interface further includes a second rotor to be rotationally driven by a second drive force transmitted from a second drive in the robot arm; and
the surgical instrument adapter further comprises:
a second driven portion to rotate a rotating portion connected to a shaft of the manual surgical instrument; and
a second drive force transmission mechanism to transmit the second drive force from the second rotor to the second driven portion.

11. The surgical instrument adapter according to claim 10, wherein the second drive force transmission mechanism includes a gearing between the second rotor and the second driven portion to transmit the second drive force from the second rotor to the second driven portion.

12. A surgical robot comprising:
a robot arm including a drive; and
a surgical instrument adapter to operably connect a manual surgical instrument to the robot arm; wherein
the surgical instrument adapter includes:
an interface including a rotor to be rotationally driven by a drive force transmitted from the drive in the robot arm;
a driven portion to rotate an operation handle of the manual surgical instrument; and
a drive force transmission mechanism to transmit the drive force from the rotor to the driven portion; and
the driven portion includes:
a lever to be rotated by the drive force; and
a roller connected to the lever, contacting the operation handle, and operable to move along a surface of the operation handle.
